(19) United States
(12) Patent Application Publication
(10) Pub. No.: US 2002/0151874 A1
(43) Pub. Date: Oct. 17, 2002

(54) Liposuction cannula device and method

(76) Inventors: Alwin H. Kolster, Anaheim, CA (US); Jeffrey A. Kolster, Lake Elsinore, CA (US); Paul J. Weber, Fort Lauderdale, FL (US); Luiz B. Da Silva, Danville, CA (US)

Correspondence Address: John P. Wooldridge, 1334 Ridgestone Court, Livermore, CA, 94551, US

(21) Appl. No.: 09/834,555

(22) Filed: Apr. 12, 2001

(51) Int. Cl. A61M 1/00

(52) U.S. Cl. 604/542

(57) ABSTRACT

A liposuction apparatus and method that uses a rotating or rotationally oscillating cannula. The advantages of the liposuction apparatus are as follows: reduced need for surgeon's arm motion and stress, reduced operating time, and increased fat volume removal per unit of instrument time use.

### BACKGROUND OF THE INVENTION

1. Field of the Invention

This invention relates to a liposuction apparatus having a cannula that rotates or rotationally oscillates to improve efficacy and reduce strain to the surgeon's arm.

2. Description of the Prior Art

Liposuction, which literally means “fat suction”, is a technique that has been modified in several ways to remove not only intact fat cells and fat globules but also, with the advent of ultrasonic liposuction, fatty fluids or fatty debris from the body by means of teasing, pulling, scraping or sonication. Liposuction can be used to reduce the volume of fat in many regions of the body, but liposuction is particularly effective in such areas as the thighs and the abdomen, which contain genetically determined fat that is less responsive to diet and exercise. Liposuction is currently a well-established plastic surgery treatment performed by surgeons as an elective operation; liposuction is one of the most common surgeries performed in the world.

There are now several main forms of liposuction used by surgeons to extract fat including: traditional, ultrasonic, reciprocating and microwave. Each of these modalities varies in its necessity or usefulness, depending upon the area of the body being treated, the amount of fibrous tissue which is mixed in with the fat to be treated, the number of times the fat has been previously suctioned (which usually increases the fibrous and resistant nature of the fat), and the genetic makeup of the individual patient (African-American and Mediterranean ancestry patients and males usually have more fibrous fat). Each modality of liposuction, again, has its benefits and drawbacks that herein follow.

In traditional liposuction, a single-lumen-annula-shaft attached to a handle, is commonly pushed and pulled by a surgeon through entrance wounds into the target fat in a spoke-wheel or radial fashion. Unfortunately, this process is difficult and can lead to surgeons having physical conditions similar to tennis elbow and arthritis of the involved joints. For this reason, a variety of mechanisms have been incorporated into liposuction cannulas to reduce the strain to the surgeon.

Ultrasonic (10-20 KHz) liposuction cannulas were developed by Parisi et al. in the late 1980's. See U.S. Pat. No. 4,886,791, titled “Liposuction Procedure With Ultrasonic Probe”, and U.S. Pat. No. 4,861,332, titled “Ultrasonic Probe”. These ultrasonic liposuction devices do facilitate removal of more fibrous fat while reducing the additional forces required for removal of the desired amount of fat. Unfortunately, ultrasonic liposuction equipment is extremely expensive, relatively difficult to set-up and use during surgery and can occasionally bore through the abdominal layers and into crucial organs. Another downside is that ultrasonic cannulas have a limited lifetime (˜30 hours). Additionally, the thermal and vibrational energy imparted by the ultrasonic device has damaged patient's nerves causing permanent debilitating back pain in many patients and has resulted in litigation. Water and attendant lines must be constantly used to cool current ultrasonic machinery while in use in the human body to avoid burning.

Motorized reciprocating liposuction systems were developed in an effort to reduce the energy required for the surgeon's arm to bore through and/or extract fibrous fat It was found relatively early on, in the late 1980's, that a 100 Hz to and fro rectilinear motion of the cannula shaft could be achieved by various motors placed about the cannula handle. Although these devices do appear to improve liposuction efficiency, the “jackhammer-like” motion of the rectilinear reciprocating liposuction devices has resulted in surgeons complaining of pain consistent with carpal tunnel syndrome in addition to elbow pain.

Prior patents have issued for the process of slicing or cutting, in which two shafts, both an inner and an outer shaft, move in an axial direction. In these devices openings align for an instant fat to protrude into the opening to be quickly cut as the two shafts slide apart closing the opening. This device unfortunately is susceptible to clogging by the fibrous septal bands that course through the fat, nourish it, and provide support.

U.S. Pat. Nos. 5,665,101 and 5,720,760 by Becker et al. describe a device with inner and outer tubular sections in which the inner tubular section rotationally oscillates to improve tissue removal. The design of this system requires that the shapes of the distal ends of the inner and outer tubular sections are relatively similar and generally rounded. However, this reduces the efficacy of this device to separate tissue planes and makes pushing the device through tissue more difficult.

Microwave liposuction systems were recently developed and are being tested for efficacy in removing fat. The tests are preliminary, but fibrous fat removal remains a concern as well as usability, possible long-term tissue effects, patient safety, and sterilization concerns. Currently many further studies need to be conducted for safety.

Given the limitations of current liposuction devices, there is a need for a novel liposuction cannula that can safely remove fat while reducing the mechanical trauma and strain experienced by the surgeon. The present invention, which uses a rotating or rotationally oscillating cannula design, fulfills this need, and further provides related advantages.

### SUMMARY OF THE INVENTION

It is an object of the present invention to provide an improved rotating or rotationally oscillating liposuction cannula apparatus and method.

A further object of the invention is to provide a rotationally oscillating liposuction cannula apparatus that utilizes a single or plurality of moving shaft(s) for slicing or cutting a desired amount of fat.

In one embodiment of the device, an electric motor in the handle of the liposuction handheld device causes a hollow shaft to rotate or rotationally oscillate within a predefined angular range (e.g. −60 to 60 degrees). A vacuum within the hollow shaft pulls fat into an opening at the distal tip of the device and out through the shaft into an external container. As the cannula rotates and is moved forward and backward by the surgeon, the opening cuts and removes body fat. The radial oscillations, which will typically be at frequencies of less than 500 Hz, will improve the cutting efficiency. In addition, more tissue is exposed to the opening during one pass increasing the changes that material will be suctioned out.

In an alternative embodiment, the device has a stationary shaft outside the oscillating shaft. In this embodiment the two shafts have openings at the distal tip that only align for a single or multiple narrow range of angles. As the internal shaft oscillates it cuts/tears the fat that enters through the opening of the first shaft. The internal shaft can have a single or multiple openings. The material between the openings acts like a scissor to cut and tear the fat.

Although embodiments herein describe that the oscillating shaft is powered by an electric motor, it would also be possible to power this device pneumatically with air or liquid.

These and other objects will be apparent to those skilled in the art based on the teachings herein. Other objects and advantages of the present invention will become apparent from the following description and accompanying drawings.

### BRIEF DESCRIPTION OF THE DRAWINGS

The accompanying drawings, which are incorporated into and form a part of the disclosure, illustrate embodiments of the invention and, together with the description, serve to explain the principles of the invention.

FIG. 1 is an illustration showing the key components of the handheld section of the rotating liposuction device.

FIG. 2 is a cross sectional view of the device of FIG. 1.

FIG. 3 is a cross-sectional view of several possible different distal tips for the rotating liposuction device.

FIG. 4 shows a complete system.

FIGS. 5A and 5B show an alternate embodiment.

FIGS. 6A and 6B show a connected and disconnected device respectively.

### DETAILED DESCRIPTION OF THE INVENTION

The present invention is directed to a liposuction apparatus and method that uses rotating or oscillating rotational motion to improve efficacy.

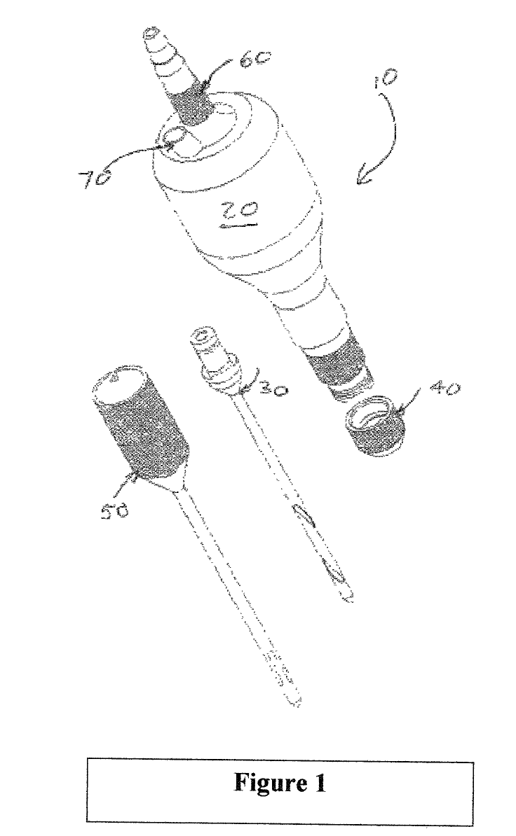

FIG. 1 shows the key components of the handheld liposuction device. The handheld device 10 has a housing 20 that is gripped by the physician during use. A removable cannula 30 connects to the motor within the housing and is locked into position with collar 40. An optional outer cannula 50 can be placed over the removable cannula 30 and attaches to the housing 20. A connector port 60 attaches via transport tube (or hose) to a vacuum pump that suctions fluids, and tissue through openings in the cannula. An electrical connector 70 is used to deliver power to the motor within the housing 20.

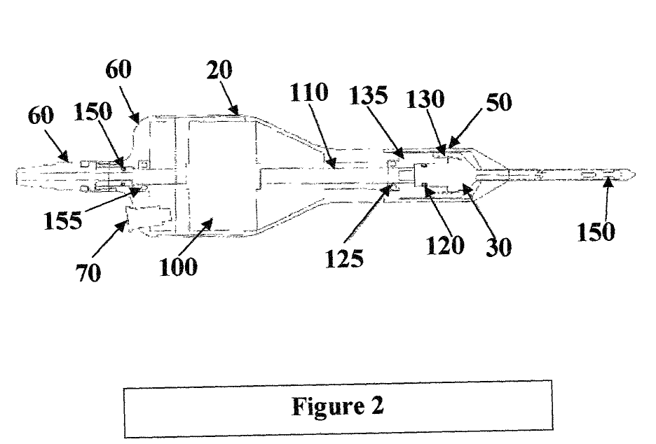

FIG. 2 shows a detailed cross sectional view through one embodiment of the handheld liposuction device 10. A housing 20 contains a motor 100 with a hollow shaft 110 that connects to a vacuum port 60 and a removable cannula 30. The motor can rotate clockwise, or counterclockwise, through multiple rotations or oscillate back and forth through a predefined range of angles (e.g., −60 to 60 degrees). An example of a suitable motor is the Hayden motor 46440-05 with hollow shaft. The removable cannula 30 attaches to one end of the shaft 110 and forms a vacuum seal at the O-ring 120. An O-ring 150 near the proximal end of the housing 20 forms the second vacuum seal. A collar 130 screws onto a cannula holder 135 to secure the inner cannula to the shaft 110. Rotary bearings 125 and 155 hold the shaft within the housing 20. A unique feature of this hollow shaft motor design is that the removed material flows easily along a straight-line path. This reduces the chances of clogging and simplifies cleaning.

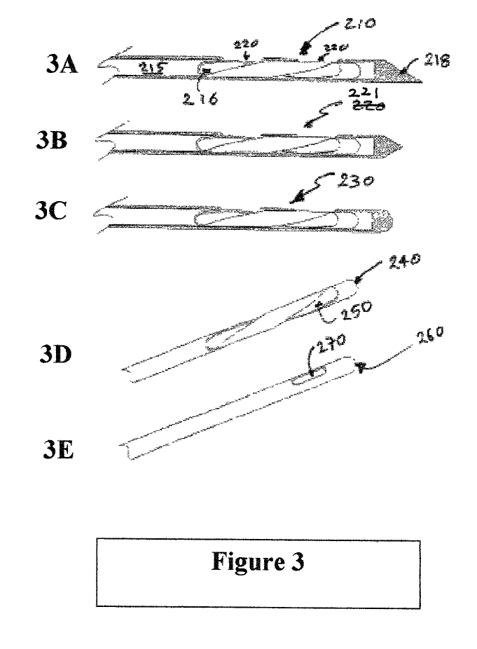

An optional second outer cannula 50 (also shown in FIG. 1) can be placed over the rotating cannula 30. The optional second cannula 50 is stationary and offers the physician the option of having an opening 150 that is directed in a well-defined direction (e.g., away from skin). The cannulas can each have one or multiple openings as illustrated in FIGS. 3A-3E. As the openings in the two cannulas slide past each other they act to cut and tear fat so that it can be easily suctioned out through the central lumen.

When the handheld liposuction device 10 is used with only the inner rotating cannula, fat and fluids are suctioned directly into the openings. The rotating action enhances tissue cutting and tearing as well as increases the volume of tissue that is exposed to suction during a pass of the device. These advantages of the rotating action make the liposuction procedure faster and potentially reduce the trauma to the physician's hand. In order to minimize the transmission of motor vibrations to the physician's hand, the housing can be covered with a high frequency absorbing material (e.g. rubber).

FIGS. 3A-3E show examples of a variety of cannula shapes that can be used with the present handheld liposuction device. In FIG. 3A, a double cannula design 210 uses an inner cannula with a spiral opening 216. The outer cannula 218 (shown in cross section) has two openings 220 at the top and a wedged tip design. The wedged tip makes it easier to separate tissue planes and move the device through the tissue. FIG. 3B shows a double cannula design 221 that has an outer cannula with a pointed tip. FIG. 3C shows a double cannula design 230 that has an outer cannula with a rounded tip that can be used when the physician wants to minimize the risk of perforating skin.

Inner cannula designs 240 of FIG. 3D and 260 of FIG. 3E show two different possible opening designs. In one design, the opening 250 has a spiral shape that applies a shearing action to enhance tissue cutting and tearing. A simpler design 260 uses a single small opening 270 that provides the physician with more control over the area being treated.

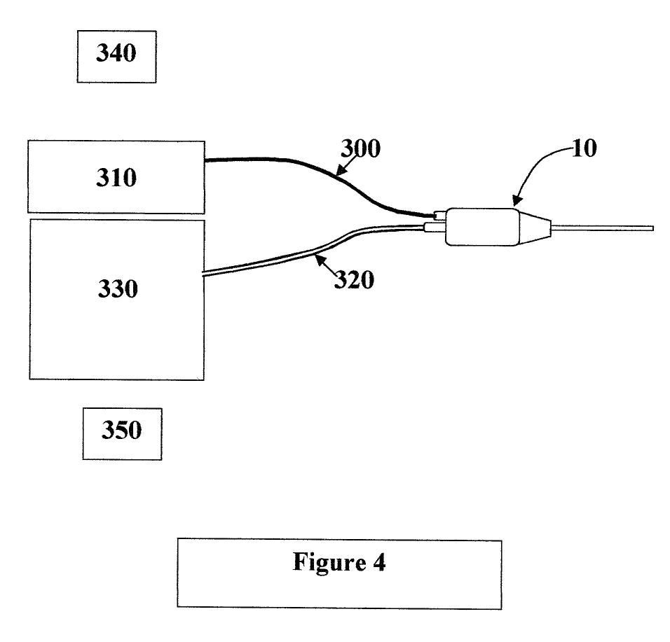

FIG. 4 shows a schematic illustration of an embodiment of the complete liposuction system. The liposuction handheld device 10 is connected through a cable 300 to an electronic control unit 310 that controls the electric motor. A user interface (e.g., switches, or touch screen) on the control unit 310 allows the user to activate the motor and specify the rotation speed, rotation type (clockwise, counterclockwise, or oscillating). An optional wireless foot switch 340 can be used to turn the motor off and on. When in oscillating rotational mode, the user can specify the center angle and angular range. A vacuum tube 320 connects the handheld device 10 to an aspiration unit. The aspiration unit includes a vacuum pump and a fluid trap that prevents tissue and fluid from reaching the vacuum pump. A user interface (e.g., switches, or touch screen) on the aspiration unit 330 allows the user to turn on the unit and specify the vacuum pressure. An optional wireless foot switch 350 can be used to tarn the aspirator on and off.

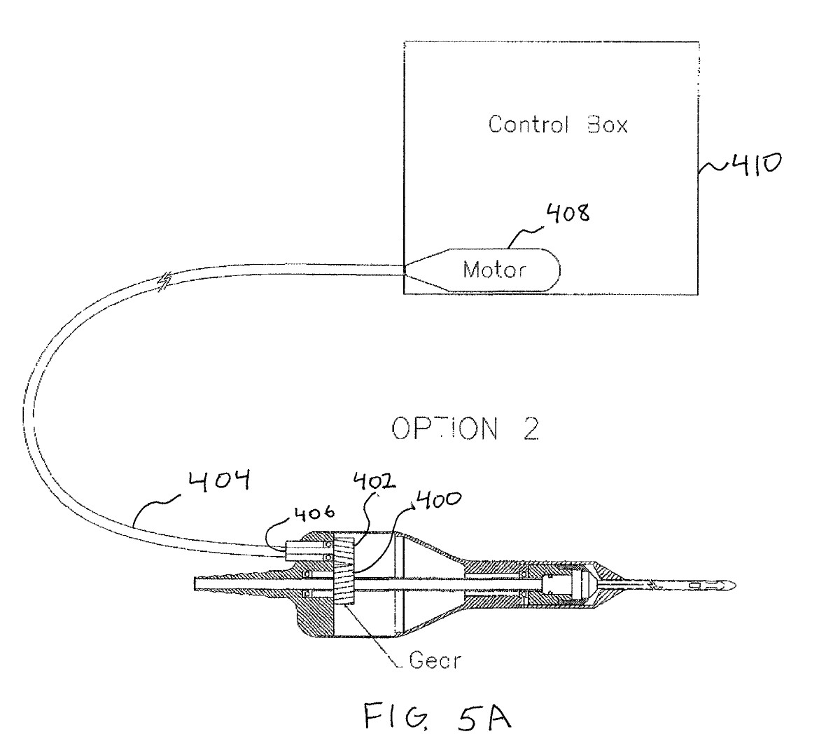

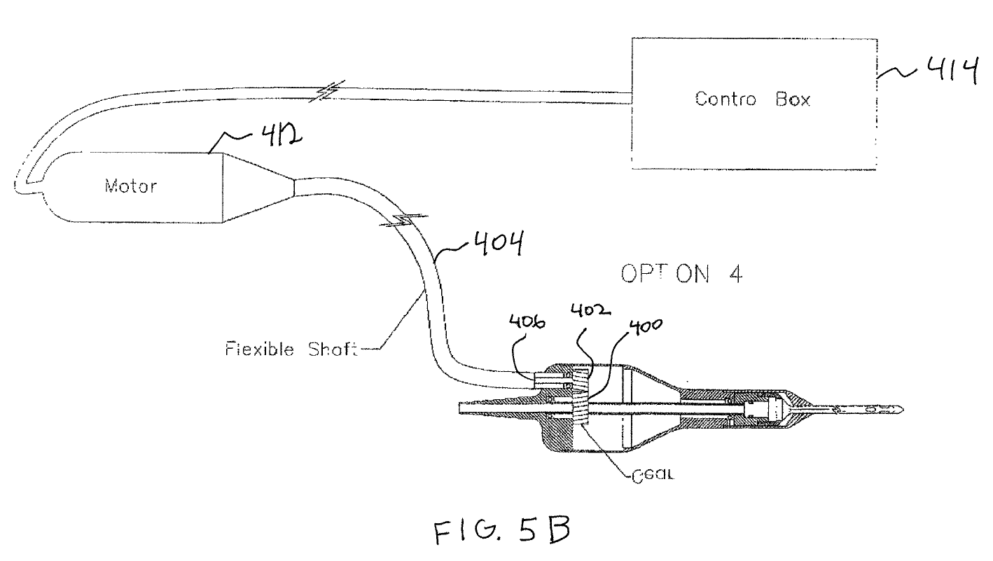

FIGS. 5A and 5B show an alternative embodiment of the handheld liposuction device where the motor that was in the handheld housing is replaced by a spur gear 400 that is driven by a second spur gear 402 (or worm gear) that is offset from the central axis. This second gear 402 is driven using a flexible cable 404 (e.g., Stock Drive Products flexible shafts) that attaches to a shaft 406 that extends through the housing. In the embodiment of FIG. 5A, the electric motor 408 is located in the external control electronic system 410. In the embodiment of FIG. 5B, the electric motor 412 is located in the external control electronic system 414. The advantage of these embodiments is that the weight of the hand piece and motor vibrations are reduced.

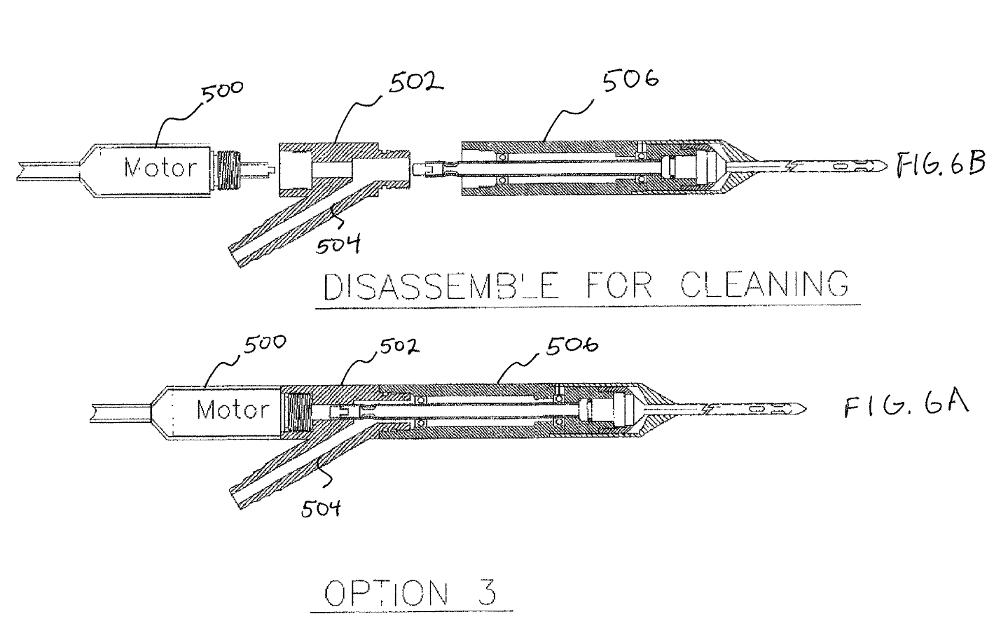

FIG. 6A illustrates a device that includes a motor 500 connected to an intermediate piece 502 that includes a passage 504. Intermediate piece 502 is connected to the endpiece 506. FIG. 6B shows motor 500, intermediate piece 502 and endpiece 506 all disconnected for cleaning.

While particular embodiments of the liposuction cannula device have been illustrated and/or described, and particular parameters have been set forth to exemplify and teach the principles of the invention, such are not intended to be lifting. Modification and changes may become apparent to those skilled in the art, and it is intended that the invention be limited only by the scope of the appended claims.

We claim:

1. A handheld liposuction device, comprising:
a single cannula;
means for holding and maneuvering said cannula; and
means for driving said cannula in a motion selected from a group consisting of a rotating motion and a rotating/oscillating motion.

2. The device of claim 1, further comprising:
a straight through suction path connected to said single cannula;
wherein said means for driving said cannula comprises a hollow shaft rotating motor, wherein said cannula comprises at least one opening, said cannula attached to said rotating motor.

3. The device of claim 1, further comprising a stationary outer cannula with a wedge or pointed tip.

4. The apparatus of claim 1, wherein said means for driving said cannula is chosen from the group consisting of an electrical motor and a pneumatic power source.

5. The apparatus of claim 1, wherein said cannula has a distal opening for aspirating fluid material through said shaft.

6. A liposuction device, comprising:
a handpiece;
a cannula operatively connected to said handpiece;
a motor located in said handpiece and operatively connected to said cannula for generating non-rectilinear rotating or rotation/oscillation motion at said distal end of said cannula;
suction means operatively connected to said cannula for aspirating fluid material through said cannula.

7. The device of claim 6, further comprising a second shaft located about said cannula for enhanced fat shearing or guidance near the entrance port.

8. The device of claim 7, wherein said motor is an electric motor.
